(19) United States
(12) Patent Application Publication
(10) Pub. No.: US 2019/0374376 A1
(43) Pub. Date: Dec. 12, 2019

(54) METHOD AND SYSTEM FOR HEATING LIMB BLOOD VESSELS

(71) Applicant: CYBERX Engineering Inc., Wilmington, DE (US)

(72) Inventors: Kirill Khaliuta, Saint Petersburg (RU); Sergei Belov, Saint Petersburg (RU)

(21) Appl. No.: 16/044,780

(22) Filed: Jul. 25, 2018

Related U.S. Application Data
(63) Continuation-in-part of application No. 16/003,211, filed on Jun. 8, 2018, now Pat. No. 10,201,195.

(51) Int. Cl. A61F 7/00

(52) U.S. Cl. CPC A61F 7/007; A61F 2007/0086; A61F 2007/0078; A61F 2007/0035; A61F 2007/0034; A61F 2007/0096; A61F 2007/0094; A61F 2007/0031; A61F 2007/0039

(57) ABSTRACT

Methods and systems for heating limb blood vessels are provided. An example system may include a wearable device comprising a body configured to be disposed on a limb of a user. The wearable device may include a battery disposed within the body. The wearable device may include a heating component disposed within the body and powered by the battery. The heating component includes a heating element configured to cover a portion of a skin of the user, the portion of the skin covering at least one blood vessel of the limb. The heating element can be configured to provide heat to the portion of the skin and of the blood vessel to cause the blood vessel to dilate. The wearable device may include a controller disposed within the body and configured to regulate a temperature of the heating element. The body can be wrapped around the limb.

### CROSS-REFERENCE TO RELATED APPLICATIONS

The present application is a Continuation-In-Part of U.S. patent application Ser. No. 16/003,211 titled “Heating System for Clothing,” filed Jun. 8, 2018. The subject matter of the aforementioned application is incorporated herein by reference for all purposes.

### TECHNICAL FIELD

This disclosure generally relates to cold weather accessories. More specifically, this disclosure relates to systems and methods for heating limb blood vessels.

### BACKGROUND

In cold weather conditions, limbs (hands, fingers, feet, toes, and so forth) are typically warmed by using conventional accessories, such as socks, gloves, mittens, and so forth. These conventional accessories may be made of natural and synthetic cloth. The conventional accessories cover the limbs to capture and preserve natural heat of limbs. Since the conventional accessories utilize solely natural heat of a human, they may not be able to maintain heat at the limbs in extremely cold weather conditions.

To address the shortcomings of the conventional cold weather accessories, heated accessories have been developed. The heated accessories include, for example, heated gloves, heated socks, and so forth. The heated accessories may include heating elements that provide heat generated by chemical reactions or electrical resistance. One of the disadvantages of the current heated accessories is that they provide more heat than needed because the heating elements cover the human body excessively. Another disadvantage of current heated accessories is that they are not comfortable when used during certain activities or work that require tactile sense in hands and fingers.

### SUMMARY

This section introduces a selection of concepts in a simplified form that are further described in the Detailed Description section, below. This summary does not identify key or essential features of the claimed subject matter and is not intended to be an aid in determining the scope of the claimed subject matter.

This disclosure is generally concerned with systems and methods for heating limb blood vessels. The present technology may provide a convenient method for warming a body of a user using one or more wearable devices placed over the user's limbs.

According to one embodiment of this disclosure, a wearable device for heating limb blood vessels is provided. The wearable device may include a body configured to be disposed on a limb of a user. The wearable device may include a battery disposed within the body. The wearable device may further include a heating component disposed within the body and powered by the battery. The heating component may include a heating element configured to cover a portion of a skin of the user. The portion of the skin covers a blood vessel of the limb. The heating element can be configured to provide heat to the portion of the skin and the blood vessel to cause the blood vessel to dilate. The wearable device may further include a controller disposed within the body and configured to regulate a temperature of the heating element.

The body of the wearable device can be configured to be wrapped around the limb. The body of the wearable device can be carried out in a shape of one of a ring, a bracelet, a wristband, or a cuff.

The body of the wearable device can be configured to be disposed on a leg of the user. The heating element can be configured to cover the portion of the skin covering at least one artery of the leg.

The body of the wearable device can be configured to be disposed on a leg of the user. The heating element can be configured to cover the portion of the skin covering at least one vein within the leg.

The body of the wearable device can be configured to be disposed on an upper arm or forearm of the user. The heating element can be configured to cover to the portion of the skin covering one or more arteries or veins within the upper arm or the forearm of the user.

The body of the wearable device can be configured to be disposed on a wrist of the user. The heating element can be configured to cover the portion of the skin covering one or more arteries or veins within the wrist of the user

The wearable device may further include a communication module disposed within the body. The communication module can be configured to be communicatively connected to a mobile device for receiving a user input. The user input may include at least a desired temperature for the heating element. The wearable device may further include an ambient temperature sensor disposed within the body and configured to measure an ambient temperature. The controller of the wearable device can be configured to receive the user input from the communication module and the ambient temperature from the ambient temperature sensor. The controller can further adjust the temperature of the heating element based on the user input and the ambient temperature.

The heating component of the wearable device may further include a temperature sensor to sense the temperature of the heating element and a power switch. The power switch can be configured to receive, from the temperature sensor, an indication that the temperature exceeds a predetermined threshold. In response to receiving, the power switch can disconnect the heating element and the battery. The heating element of the heating component may include a copper conductor.

According to another embodiment of this disclosure, a method for heating limb blood vessels is provided. An example method may include providing a body made of a flexible material and configured to be disposed on a limb of a user. The method may include disposing a battery within the body. The method may further include disposing, within the body, a heating component powered by the battery. The heating component may include a heating element configured to cover a portion of skin of the user. The portion of the skin covers a blood vessel of the limb. The method may include disposing, within the body, a controller operable to regulate a temperature of the heating element to provide heat to the portion of the skin and the blood vessel to cause the blood vessel to dilate.

Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities, and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 is a block diagram showing a system for heating limb blood vessels, according to some example embodiment of the present disclosure.

FIG. 2 shows example wearable devices for heating limb blood vessels disposed on a body of a user 200, according to one example embodiment of the present disclosure.

FIG. 3 shows wearable devices for heating limb blood vessels on a right arm of a human, according to an example embodiment.

FIG. 4 shows wearable devices for heating limb blood vessels on a right leg of a human, according to an example embodiment.

FIG. 5 is a block diagram of a heating component of a wearable device, according to some example embodiments of the present disclosure.

FIG. 6 is a block diagram of a control plate of a wearable device, according to some example embodiments of the present disclosure.

FIG. 7 is flow chart showing steps of a method for heating limb blood vessels, according to some example embodiments of the present disclosure.

### DETAILED DESCRIPTION

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

Embodiments of this disclosure are concerned with systems and methods for heating limb blood vessels. Embodiments of this disclosure may provide a method for warming a body of a user by disposing one or more wearable devices around the user's limbs.

According to an example embodiment, the wearable device may include a body configured to be disposed on a limb of a user. The wearable device may include a battery disposed within the body. The wearable device may further include a heating component disposed within the body and powered by the battery. The heating component may include a heating element configured to cover a portion of a skin of the user. The portion of the skin covers a blood vessel of the limb. The heating element can be configured to provide heat to the portion of the skin and the blood vessel to cause the blood vessel to dilate. The wearable device may further include a controller disposed within the body and configured to regulate a temperature of the heating element.

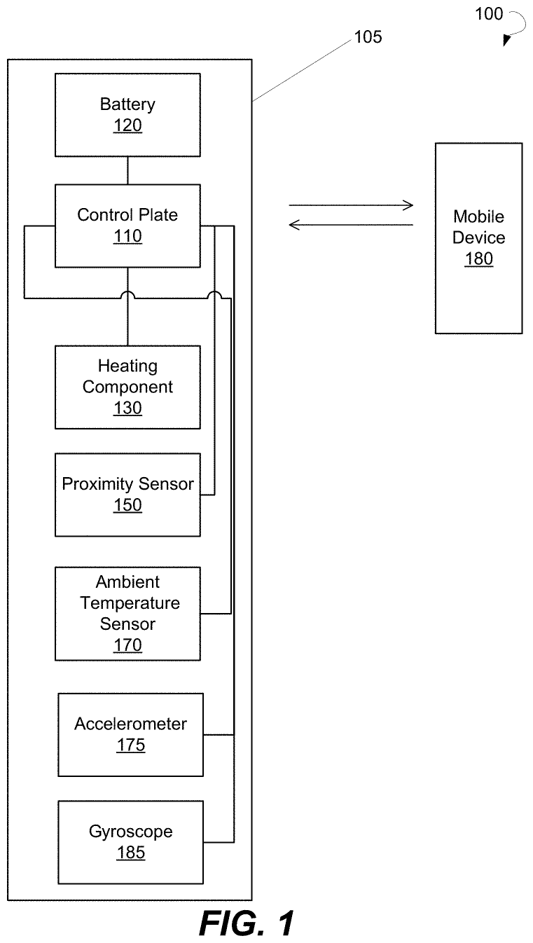

FIG. 1 is a block diagram of system 100 for heating limb blood vessels, according to some example embodiments of the present disclosure. The system 100 may include one or more wearable device(s) 105 and a mobile device 180.

The wearable device 105 can be carried in a shape of a bracelet, a wristband, a cuff, a ring, a bracelet, and the like. The wearable device 105 can be positioned around a limb of a user, for example an arm or a leg. In some embodiments, the wearable device 105 may have a clasp, a hook-and-loop fastener, closure buckle, or other device to secure the wearable device 105 around the limb. In other embodiments, the wearable device 105 may include a portion made of stretch fabric to allow the wearable device 105 to be secured at a desired spot around the limb of the user.

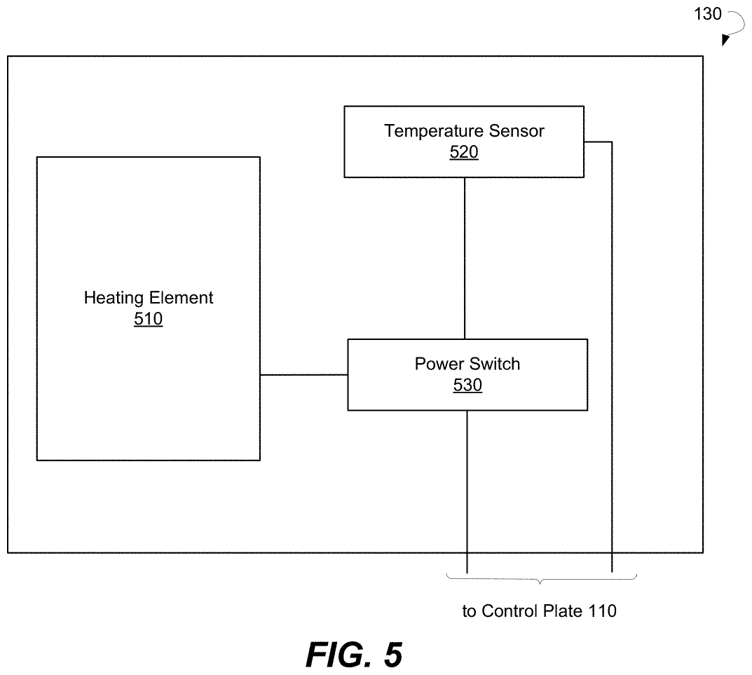

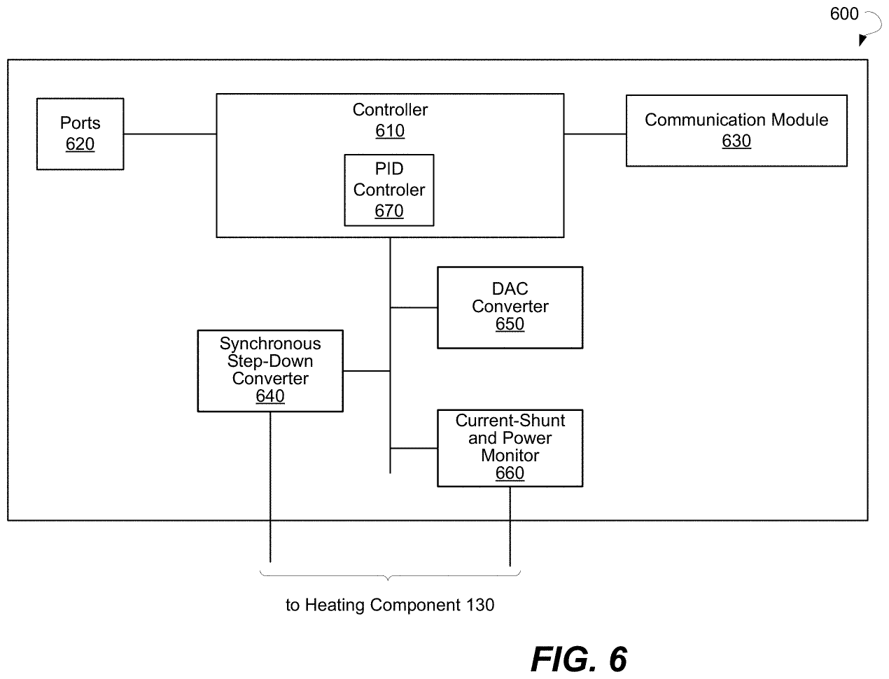

The wearable device may include a control plate 110, a battery 120, a heating component 130, a proximity sensor 150, and an ambient temperature sensor 170. The wearable device 105 may further include an accelerometer 175 and a gyroscope 185. Details of an example heating component 130 are described further in FIG. 5. Details of an example control plate 120 are described further in FIG. 6. In a further embodiment, the wearable device 105 may include other components required for operation of the wearable device 105. For example, the wearable device 105 may further include a touchscreen input device for receiving a user input. The user input may include, for example, an operational mode and a desired temperature for the heating component 130.

The mobile device 180 can be communicatively coupled with the control plate 110 of the wearable device 105 by a wireless communication protocol, such as Bluetooth™. The mobile device 180 may include a smartphone, a tablet computer, a smart watch, and the like. A user of the wearable device 105 may use an application of the mobile device 180 to provide a user input (for example, an operational mode and a desired temperature for the heating component 130). In addition, the user may use the application of the mobile device 180 to monitor temperature of the heating component 130.

The heating component 130 can be disposed on an inner surface of the wearable device 105, which touches the user body when the wearable device 105 is disposed on the user limb.

The proximity sensor 150 may include a capacitive proximity sensor. The proximity sensor 150 can be disposed on the inner surface of the wearable device 105 and configured to detect whether the wearable device 105 touches a body of the user (that is, whether the wearable device 105 is disposed on user body).

The ambient temperature sensor 170 can be disposed on an outer surface of the wearable device 105, which is opposite to the inner surface and does not touch the user's body when the wearable device 105 is disposed in the user's limb. The ambient temperature sensor 170 can be configured to sense a temperature of ambient air.

The accelerometer 175 may include a 3-axis accelerometer. The accelerometer 175 may provide data on proper acceleration of the wearable device 105. The gyroscope 185 can provide data on orientation and angular velocity of the wearable device 105. The data from the accelerometer 175 and gyroscope 185 may be provided to the control plate 110 via an integrated circuit (I²C) bus.

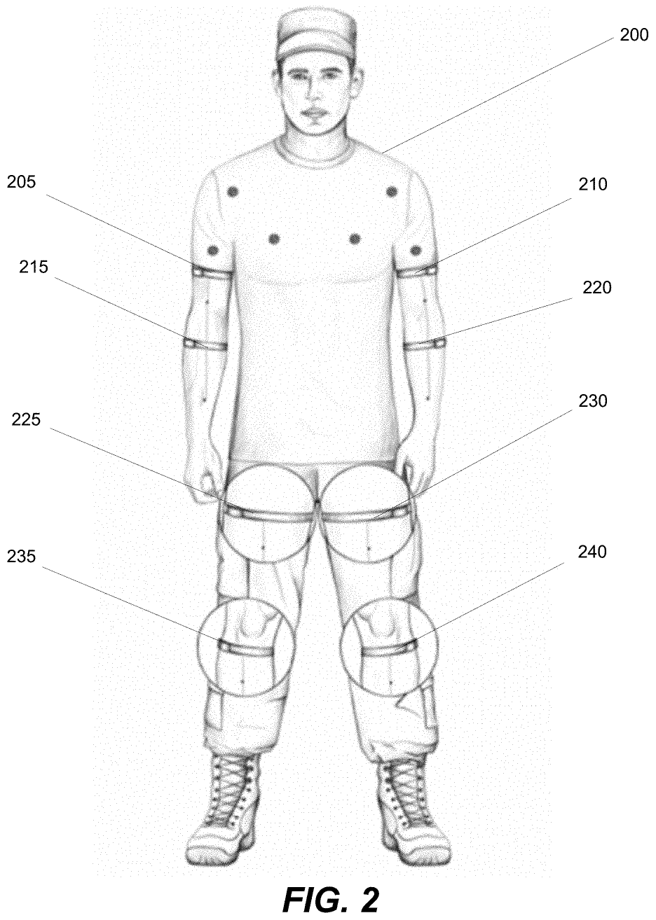

FIG. 2 shows example wearable devices 205, 210, 215, 220, 225, 230, 235, and 240 for heating limb blood vessels disposed on a body of a user 200, according to an example embodiment of the present disclosure. The wearable devices 205-240 may include components analogous to the wearable device 105 described in FIG. 1. The wearable devices 205-240 may differ in size from each other. The specific sizes of the wearable devices 205-240 may be consistent with a circumference of a limb of the user at a desired spot at which the wearable device is supposed to be disposed.

As shown in FIG. 2, the wearable device 205 may be positioned on a right upper arm of the user 200. The wearable device 210 can be positioned on a left upper arm of the user 200. The wearable device 215 may be positioned on a right forearm of the user 200 below the elbow. The wearable device 220 may be positioned on a left forearm of the user 200 below the elbow.

As shown in FIG. 2, the wearable device 225 may be positioned on a right thigh of the user 200. The wearable device 230 can be positioned on a left thigh of the user 200. The wearable device 235 may be positioned on a right lower leg below the right knee. The wearable device 240 can be positioned on a left lower leg below the left knee.

The wearable devices 205-240 can be rotated around the limb in such a way that heating elements of the wearable devices 205-240 would cover portions of human tissue (skin and muscles) covering blood vessels. In a cold weather, the blood vessels supplying warm blood to limbs can become constrict. The constriction of the blood vessels is a natural reaction that reduces the flow of warm blood near the surface of the skin, thereby reducing heat loss. At the same time constriction of the blood vessels reduces amount of warm blood provided to the limbs, so the limbs and, especially, hands, feet, and fingers may become cold. The heat provided by the heating elements to the portion of the human tissue and the blood vessels covered by the human tissue causes a response by the autonomic nervous system to dilate the blood vessels. When the blood vessels dilate, the amount of warm blood provided to the limbs increases. It is preferable to dispose the heating elements against veins, rather than arteries, since warming veins is more effective with regards to power consumption than warming the arteries.

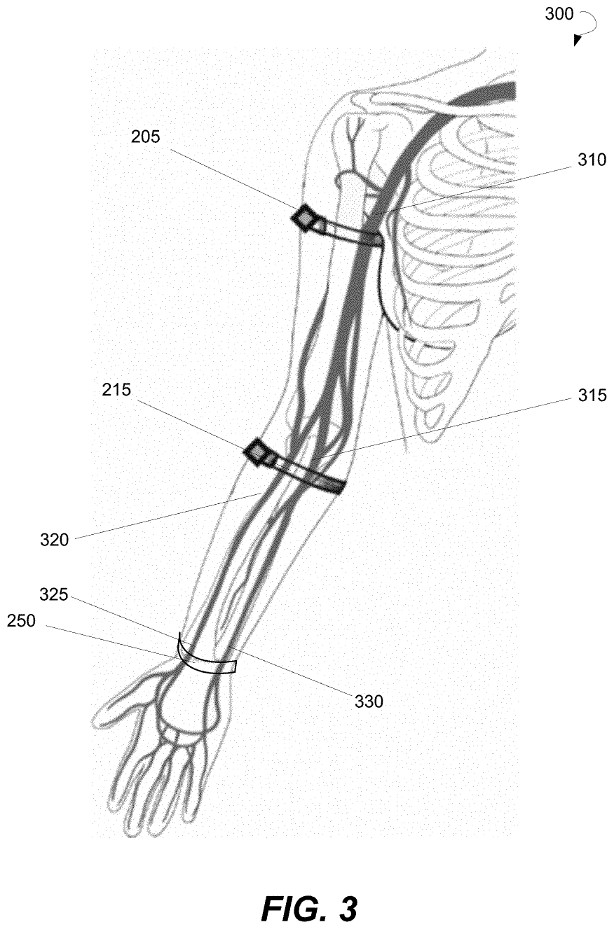

FIG. 3 shows example wearable devices 205, 215, and 250 for heating limb blood vessels disposed on a right arm of a user 200, according to an example embodiment. The wearable device 205 can be disposed around the right upper arm. The wearable device 205 can be positioned in a such way that the heating component of the wearable device 205 touches a portion of human tissue against one or more blood vessels within the right upper arm. For example, the heating component of the wearable device 205 may be positioned against axillary vein 310 of the user. In such position, the heating component of the wearable device may provide heat to the axillary vein.

The wearable device 215 can be disposed around the right forearm of the user. The wearable device 215 can be positioned in a such way that the heating component of the wearable device 215 touches a portion of human tissue against one of the blood vessels within the right forearm. For example, the heating component of the wearable device 215 may be positioned above basilic vein 315 and/or cephalic vein 320 of the user. In such position, the heating component of the wearable device may provide heat to the basilic vein 315, cephalic vein 320, or both.

The wearable device 250 can be disposed around the right wrist of the user. The wearable device 250 can be rotated around the right wrist to dispose heating component of the wearable device 250 against one or more blood vessels of the wrist (for example, a radial artery 325 and/or ulnar artery 330). Disposing the heating element of the wearable device 250 may allow to provide heat to the arteries of the right wrist causing the arteries to dilate. When the arteries of the right wrist dilate, they provide more blood flow to blood vessels of fingers and the rest of the right hand of the user.

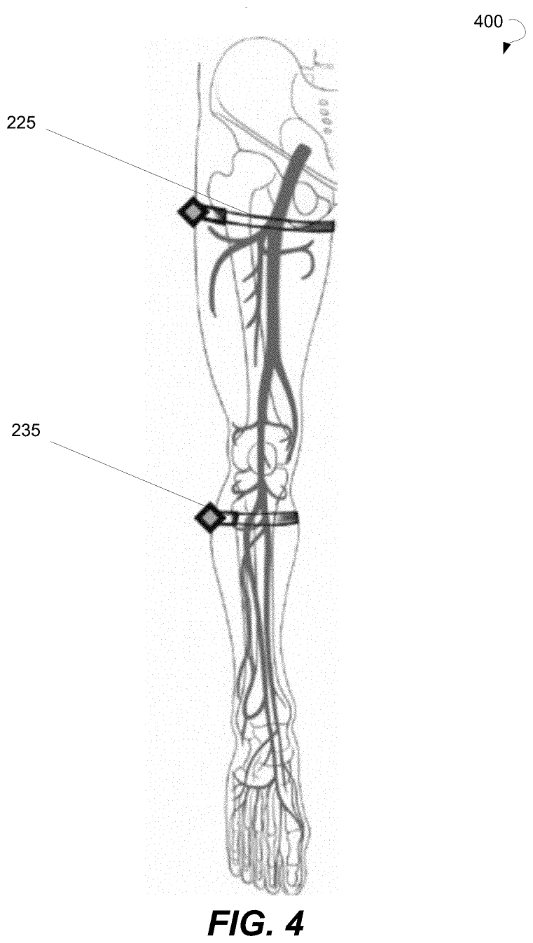

FIG. 4 shows example wearable devices 225 and 235 for heating limb blood vessels disposed on a right leg of the user 200, according to an example embodiment.

The wearable device 225 can be disposed around right thigh of the user 200. The wearable device 225 can be rotated around the right thigh to dispose the heating component of the wearable device 225 against desirable blood vessels of the left thigh. For example, the heating component of the wearable device 225 can be disposed against the great saphenous vein, femoral vein, small saphenous vein, and perforating veins. The heating component of the wearable device 225 can be disposed to cover one of the arteries within the left thigh (for example, femoral artery).

The wearable device 235 can be disposed around right lower leg of the user 200 below the right knee. The wearable device 235 can be rotated around the right lower leg to dispose the heating component of the wearable device 235 against desirable blood vessels of the right lower leg. For example, the heating component of the wearable device 235 can be disposed against the great saphenous vein, small saphenous vein, and/or anterior tribal vein. The heating component of the wearable device 235 can be also disposed to cover one of the arteries within the right lower leg (for example, anterior tibial artery, posterior tibial artery, and peroneal artery). Due to heat the arteries may dilate and provide more blood flow to the blood vessels of the right foot of user 200.

FIG. 5 is a block diagram of example heating component 130 of the wearable device 105, according to example embodiments of the present disclosure. The heating component 130 may include a heating element 510, a temperature sensor 520, and a power switch 530.

In an example embodiment, the heating element 510 may include a copper conductor. The thickness of the copper conductor can be 35 micrometers. The heating component 130 may also include buses for providing power and signals to the heating element 510, the temperature sensor 520, and the power switch 530. For example, the temperature sensor 520 and the power switch 530 can be connected using an I²C bus. The heating element 510, the temperature sensor 520, the power switch 530 can be disposed on a flexible plate. The flexible plate can be made of polyamide. The thickness of the polyamide can be 50 micrometers. The heating component 130 can be made in one technological cycle using the same technology as used for manufacturing flexible printed circuit boards.

The temperature sensor 520 may be in a thermal contact with heating element 510 and may be configured to provide data to the power switch 530 and the control plate 110. The data can be provided using an I²C circuit bus. The temperature sensor 520 may include an open-drain EVENT output used to indicate an alarm condition in which the measured temperature exceeds a user-programmable pre-determined threshold. The pre-determined threshold can be set to 85 degrees of Celsius.

The power switch 530 can be operable to disconnect and connect the heating element 510 from a power source provided by the control plate 110. The power switch 530 may include a multichannel metal-oxide-semiconductor transistor. The power switch 530 can be connected directly to temperature sensor 520 to receive the indication of alarm condition. Upon receiving the indication of alarm condition, the power switch 530 can disconnect the heating element 510 from the power source. It should be noted that the power switch 530 may disconnect the heating element 510 from the power source solely based on the indication of alarm from the temperature sensor 520 independent from any signals from the control plate 110.

The power switch 530 may also be configured to receive further indications of alarms (for example, indication that amperage exceeded a predetermined value maximum). The further indications can be received from the control plate 110.

FIG. 6 is a block diagram of a control plate 600, according to one example embodiment of the present disclosure. The control plate 600 may include a controller 610, one or more ports 620, and a communication module 630. The control plate 110 may further include a synchronous step-down converter 640, a digital-to-analog converter (DAC) 650, and a current-shunt and power monitor 660.

The controller 610 and communication module 620 may be configured to receive power from the battery 120. The control plate 110 may be carried out as a printed circuit board made of a glass-reinforced epoxy laminate material, such as FR-4.

The controller 610 may include a system-on-chip, system-on-modules memory, an application-specific integrated circuit, and so forth. For example, the controller 610 may include an Advanced Reduced Instruction Set Machine (ARM) microcontroller carried out on base of Cortex-M4 kernel, such as STM32F745. The controller 610 may include modules for controlling temperature heating components 130. For example, the controller 610 may include a proportional-integral-derivative (PID) controller 670. The PID controller 670 can control output voltage provided to the heating component 130.

The synchronous step-down converter 640 can be used to regulate output voltage provided to the heating component 130. The synchronous step-down converter 540 may operate at a switching frequency of 2.5 Megahertz. In some embodiments, synchronous step-down converter TPS62135 of Texas Instruments can be used. The DAC 650 can be used for setting required value for the output voltage of the synchronous step-down converter 640. In some embodiments, DAC081S101 8-Bit Micro Power Digital-to-Analog Converter of Texas Instruments can be used as the DAC converter 650.

The current-shunt and power monitor 660 can be used to monitor amperage and voltage consumed by heating element of heating component 130. The current-shunt and power monitor 660 may include an output port to report the amperage and the voltage to the controller 310 to the controller 610. An I²C bus can be used for reporting amperage and voltage to the controller 610. The current-shunt and power monitor 660 may also include an output for indication of an alarm condition in which the monitored amperage exceeds a user-programmable pre-determined limit. The output from the current-shunt and power monitor 660 may be directly connected to the power switch 530 of the heating component 130, so the power switch 530 may disconnect the heating element 510 from the power source (synchronous step-down converter 640) when the amperage exceeds the pre-determined limit without involving the controller 610. The output of the of the current-shunt and power monitor 660 may be connected with the open-drain EVENT output of the temperature sensor 520 using an OR gate, so the power switch 530 may receive indications on alert conditions via one input. In some embodiments, INA231 current-shunt and power monitor of Texas Instruments can be used.

The ports 620 may include PCB connectors. For example, Molex SlimStack™ Hybrid Power connectors, which have both power and signal contacts, can be used. The ports 620 may be used to transfer power to the heating component 130 and other components of the wearable device 105. The ports 620 can be also used to receive and send data from temperature sensor 520, power switch 530, accelerometer 175, gyroscope 185, the ambient temperature sensor 170, and the proximity sensor 150. The ports 320 may be also used to connect the control plate 110 to the battery 120.

The controller 610 can be configured to receive, via the ports 620, an ambient temperature from the ambient temperature sensor 170. The controller 610 can be also configured to receive user-defined temperature from the mobile device 180 via communication module 630 using a wireless protocol. The controller 610 may be further configured to adjust an output voltage of the voltage of synchronous step-down converter 640 based on the user-defined temperature, the ambient temperature, and the temperature from the temperature sensor 520.

The controller 610 may be further configured to receive data from proximity sensor 150. Based on the data from the proximity sensor 150, the controller may determine that the wearable device 105 is not worn by a user. The controller 610 may further reduce the output voltage of the voltage converters to a minimum value to save battery charge.

The controller 610 may be further configured to receive data from accelerometer 175 and gyroscope 185 and determine, based on the data from the accelerometer and gyroscope, a level of physical activity of a user wearing the wearable device. The level of physical activity of the user can depend on whether user is standing, walking, running, makes movement with his hands or legs, and so forth. The controller 610 may further adjust the output voltage of the synchronous step-down converter 640 based on the level of activity. As a user is more active, less power can be provided to the heating components 130, so the user may feel more comfortable when he walks, runs, exercises, or performs work. When the user remains standing for a pre-determined period of time, the controller 610 may provide more power to the heating component 130 to increase temperature of heating element 510.

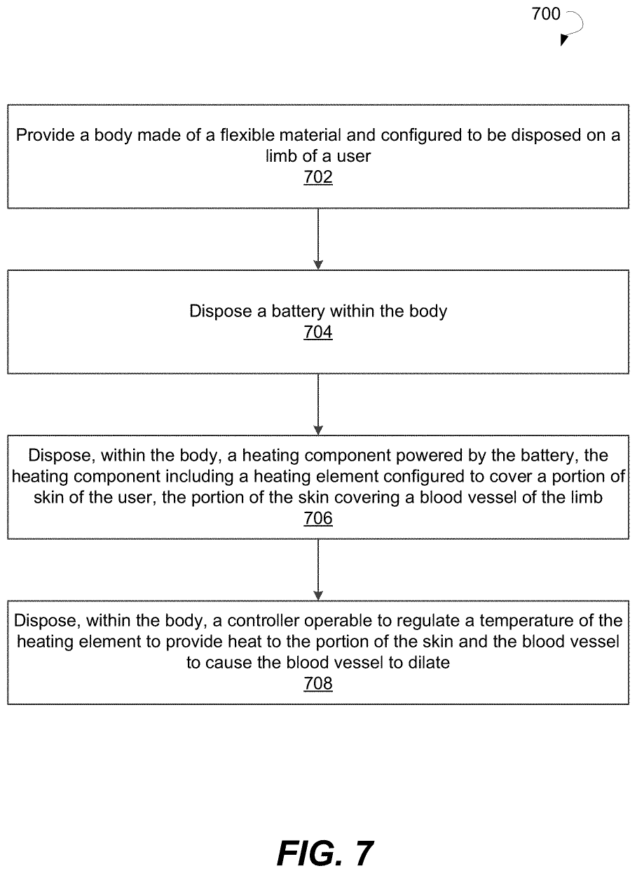

FIG. 7 is a flow chart showing a method 700 for heating limb blood vessels, according to an example embodiment. Operations of the method 700 may be implemented in an order different than described and shown in FIG. 7. Moreover, method 700 may have additional operations not shown herein, but which can be evident for those skilled in the art from the present disclosure.

The method 700 may commence, in block 702, with providing a body made of a flexible material and configured to be disposed on a limb of a user. The body can be configured to be wrapped around the limb. The body can be carried out in a shape of one of a ring, a bracelet, a wristband, and a cuff.

In block 704, the method 700 may include disposing, within the body, a battery.

In block 706, the method 700 may include disposing, within the body, a heating component powered by the battery. The heating component may include a heating element configured to cover a portion of skin of the user. The portion of the skin covers a blood vessel of the limb. The heating element can be made of copper conductor.

In block 708, the method 700 may include disposing, within the body, a controller operable to regulate a temperature of the heating element to provide heat to the portion of the skin and the blood vessel to cause the blood vessel to dilate.

Thus, the systems and methods for heating limb blood vessels have been described. Although embodiments have been described with reference to specific example embodiments, it will be evident that various modifications and changes can be made to these example embodiments without departing from the broader spirit and scope of the present document. Accordingly, the specification and drawings are to be regarded in an illustrative rather than a restrictive sense.

1. A wearable device for heating limb blood vessels, the wearable device comprising:
a body configured to be disposed on a limb of a user;
a battery disposed within the body;
a heating component disposed within the body and powered by the battery, the heating component comprising:
a heating element configured to cover a portion of a skin of the user, the portion of the skin covering a blood vessel of the limb, the heating element being configured to provide heat to the portion of the skin and the blood vessel to cause the blood vessel to dilate;
a temperature sensor to sense a temperature of the heating element; and
a power switch configured to disconnect, based on the temperature of the heating element, the heating element and the battery, and wherein the heating element, the temperature sensor, and the power switch are disposed on a flexible plate and the heating component is manufactured as a flexible printed circuit board in a single technological cycle; and
a controller disposed within the body and configured to regulate the temperature of the heating element by controlling a voltage provided to the heating component.

2. The wearable device of claim 1, wherein the body is configured to be wrapped around the limb.

3. The wearable device of claim 2, wherein the body is carried out in a shape of one of a ring, a bracelet, a wristband, and a cuff.

4. The wearable device of claim 1, wherein:
the body is configured to be disposed on a leg of the user; and
the heating element is configured to cover the portion of the skin covering at least one artery of the leg.

5. The wearable device of claim 1, wherein:
the body is configured to be disposed on a leg of the user; and
the heating element is configured to cover the portion of the skin covering at least one vein within the leg.

6. The wearable device of claim 1, wherein:
the body is configured to be disposed on an upper arm or forearm of the user; and
the heating element is configured to cover to the portion of the skin covering one or more of arteries or veins within the upper arm or the forearm of the user.

7. The wearable device of claim 1, wherein:
the body is configured to be disposed on a wrist of the user;
the heating element is configured to cover the portion of the skin covering one or more arteries or veins within the wrist of the user

8. The wearable device of claim 1, further comprising:
a communication module disposed within the body and configured to be communicatively connected to a mobile device for receiving a user input, the user input including at least a desired temperature for the heating element; and
an ambient temperature sensor disposed within the body and configured to measure an ambient temperature; and
wherein the controller is configured to:
receive the user input from the communication module and the ambient temperature from the ambient temperature sensor; and
adjust, based on the user input and the ambient temperature, the temperature of the heating element.

9. The wearable device of claim 1, wherein the power switch configured to:
receive, from the temperature sensor, an indication that the temperature exceeds a predetermined threshold; and
in response to receiving, disconnect the heating element and the battery.

10. The wearable device of claim 1, wherein the heating element includes a copper conductor.

11. A method for heating limb blood vessels, the method comprising:
providing a body made of a flexible material and configured to be disposed on a limb of a user;
disposing, within the body, a battery;
disposing, within the body, a heating component powered by the battery, the heating component comprising:
a heating element configured to cover a portion of skin of the user, the portion of the skin covering a blood vessel of the limb;
a temperature sensor to sense a temperature of the heating element; and
a power switch configured to disconnect, based on the temperature of the heating element, the heating element and the battery, and wherein the heating element, the temperature sensor, and the power switch are disposed on a flexible plate and the heating component is manufactured as a flexible printed circuit board in a single technological cycle; and
disposing, within the body, a controller operable to regulate the temperature of the heating element by controlling a voltage provided to the heating component to provide heat to the portion of the skin and the blood vessel to cause the blood vessel to dilate.

12. The method of claim 11, wherein the body is configured to be wrapped around the limb.

13. The method of claim 12, wherein the body is carried out in a shape of one of a ring, a bracelet, a wristband, and a cuff.

14. The method of claim 11, wherein:
the body is configured to be disposed on a leg of the user; and
the heating element is configured to cover the portion of the skin covering at least one artery of the leg.

15. The method of claim 11, wherein:
the body is configured to be disposed on a leg of the user; and
the heating element is configured to cover the portion of the skin covering at least one vein within the leg.

16. The method of claim 11, wherein:
the body is configured to be disposed on an upper arm or forearm of the user; and
the heating element is configured to cover the portion of the skin covering one or more arteries or veins within the upper arm or the forearm of the user.

17. The method of claim 11, wherein:
the body is configured to be disposed on a wrist of the user;
the heating element is configured to cover the portion of the skin covering one or more arteries or veins within the wrist of the user.

18. The method of claim 11, further comprising:
disposing, within the body, a communication module configured to be communicatively connected to a mobile device for receiving a user input, the user input including at least a desired temperature for the heating element;
disposing, within a body an ambient temperature sensor to measure an ambient temperature; and
configuring the controller to:
receive the user input from the communication module and the ambient temperature from the ambient temperature sensor; and
adjust, based on the user input and the ambient temperature, the temperature of the heating element.

19. The method of claim 11, further comprising:
disposing, within the body, the power switch configured to:
receive, from the temperature sensor, an indication that the temperature exceeds a predetermined threshold; and
in response to receiving, disconnect the heating element and the battery.

20. A wearable device for heating limb blood vessels, the wearable device comprising:
a flexible body configured to be wrapped around a limb of a user, the flexible body carried out in a shape of one of a ring, a wristband, a bracelet, a cuff;
a battery disposed within the flexible body;
a heating component disposed within the flexible body and powered by the battery, the heating component comprising:
a heating element configured to cover a portion of skin of the user, the portion of the skin covering a blood vessel of the limb, the heating element being configured to provide heat to the portion of skin and the blood vessel to cause the blood vessel to dilate;
a temperature sensor to sense a temperature of the heating element; and
a power switch configured to disconnect, based on the temperature of the heating element, the heating element and the battery, and wherein the heating element, the temperature sensor, and the power switch are disposed on a flexible plate and the heating component is manufactured as a flexible printed circuit board in a single technological cycle;
a communication module disposed within the flexible body and configured to be communicatively connected to a mobile device for receiving a user input, the user input including at least a desired temperature for the heating element;
an ambient temperature sensor disposed within the flexible body and configured to measure an ambient temperature;
a controller disposed within the flexible body and configured to:
receive the user input from the communication module and the ambient temperature from the ambient temperature sensor; and
adjust, based on the user input and the ambient temperature, the temperature of the heating element by controlling a voltage provided to the heating component;
a temperature sensor disposed within the flexible body and configured to sense the temperature of the heating element; and
a power switch disposed within the flexible body configured to:
receive, from the temperature sensor, an indication that the temperature exceeds a predetermined threshold; and
in response to receiving, disconnect the heating element and the battery.
